(19) United States
(12) Patent Application Publication
(10) Pub. No.: US 2018/0002021 A1
(43) Pub. Date: Jan. 4, 2018

(54) HEAD SUPPORT ADAPTABLE TO SEAT BACKS

(71) Applicant: Osborne E. Smith, Saint Albans, MO (US)

(72) Inventor: Osborne E. Smith, Saint Albans, MO (US)

(21) Appl. No.: 15/635,481

(22) Filed: Jun. 28, 2017

Related U.S. Application Data
(60) Provisional application No. 62/357,538, filed on Jul. 1, 2016.

(51) Int. Cl. B64D 11/06; A47C 7/38; A61F 9/04; B60N 2/48

(52) U.S. Cl. CPC B64D 11/062; B60N 2/4879; A47C 7/383; A61F 9/04; B60N 2002/4888

(57) ABSTRACT

A head support apparatus is provided for supporting a person's head while sitting in an airplane seat. The apparatus comprises a strap member for encircling around the back of a seat while retaining the user's head. The strap can be made adjustable and stretchable to accommodate the size of the person's head and to provide an appropriate level of snugness. The head support apparatus may provide a sleep mask element.

### RELATED APPLICATION

The present non-provisional patent application claims priority benefit of an earlier-filed provisional patent application of the same title, Serial No. 62/357,538, filed Jul. 1, 2016. The identified earlier-filed application is hereby incorporated by reference into the present application.

### FIELD OF THE INVENTION

The present invention relates to devices to provide comfort to a traveler. More specifically, the invention relates to devices that provide support to one's head while sitting in a high-backed seat, such as an airplane seat.

### BACKGROUND OF THE INVENTION

Airplane travelers must sometimes remain seated during their flights for extended periods of time. Airplane seats, especially those in coach class, are notorious for providing cramped and confined space in which a person must sit. Except for those individuals that are able to physically contort their bodies into unnatural positions, most people are limited to maintaining a relatively upright sitting position in the chair. Apart from the ability to partially recline the seat back, airplane seats do not offer the passenger adequate space that is conducive to napping.

If one does happen to nap while sitting upright in an airplane seat, there is no structure presently available on the seat that provides adequate head support. Accordingly, a napper's head may loll about, which completely interferes with any kind of relaxing sleep. A pillow provides some support to the rear of the head, and the side of the head to a degree, but provides inadequate support to prevent the head from nodding or jerking forward.

It would therefore be desirable to provide a support for a person's head to gently hold the head in place from lolling about when sitting upright in an airplane seat. It would be further desirable to provide such a support that can be easily and temporarily attached to an airplane seat. It would be further desirable to provide such a support that is simple in structure such that it can be attached to an airplane seat without the need for any separate connecting elements. It would be further desirable to provide such a support that provides a mask element that can keep one's eyes shielded from light to help induce sleep.

### SUMMARY OF THE INVENTION

The present invention comprises a head support device that permits a person to keep his/her head in a relatively upright position as he sits in an airplane seat. The device comprises a strap member of sufficient length to slip over and encircle an upper portion of the seat. The length of the strap should be long enough to permit one's head to be placed within the strap while it is engaged with the seat. The strap can be made adjustable to accommodate the size of the person's head and to provide an appropriate level of snugness. The strap can be made stretchable so that the user's head is not restricted from necessary movement.

An embodiment of the head support device may comprise a portion of the strap having a sleep mask element that the user can position over his eyes.

These and other features of the present invention are described in greater detail below in the section titled DETAILED DESCRIPTION OF THE INVENTION.

### BRIEF DESCRIPTION OF THE DRAWING FIGURES

The present invention is described herein with reference to the following drawing figures, with greater emphasis being placed on clarity rather than scale:

FIG. 1 is a perspective view of the invention.

FIG. 2 is a view of a person sitting in an airplane seat while wearing the invention.

FIG. 3 is a top plan view of the invention.

FIG. 4 is a view in side elevation of the invention taken along the line 4-4 in FIG. 3.

### DETAILED DESCRIPTION OF THE INVENTION

With reference to the drawings figures, an embodiment of the present invention is herein described, shown, and otherwise disclosed.

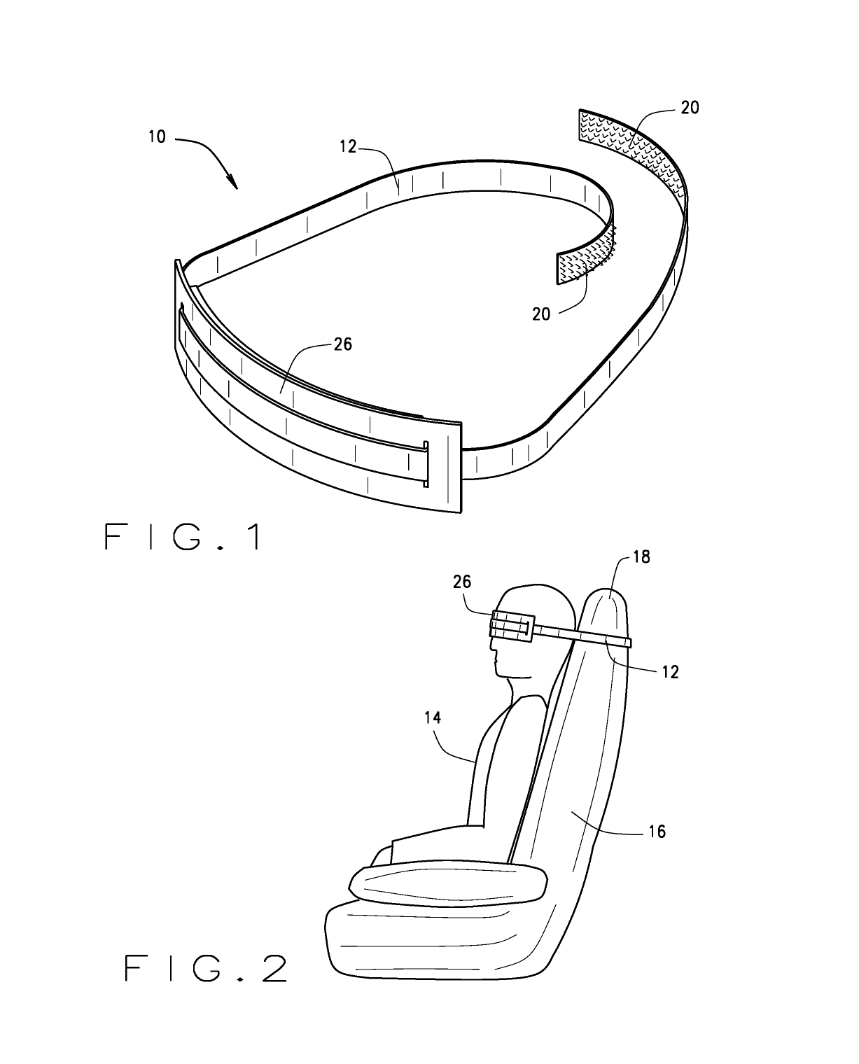

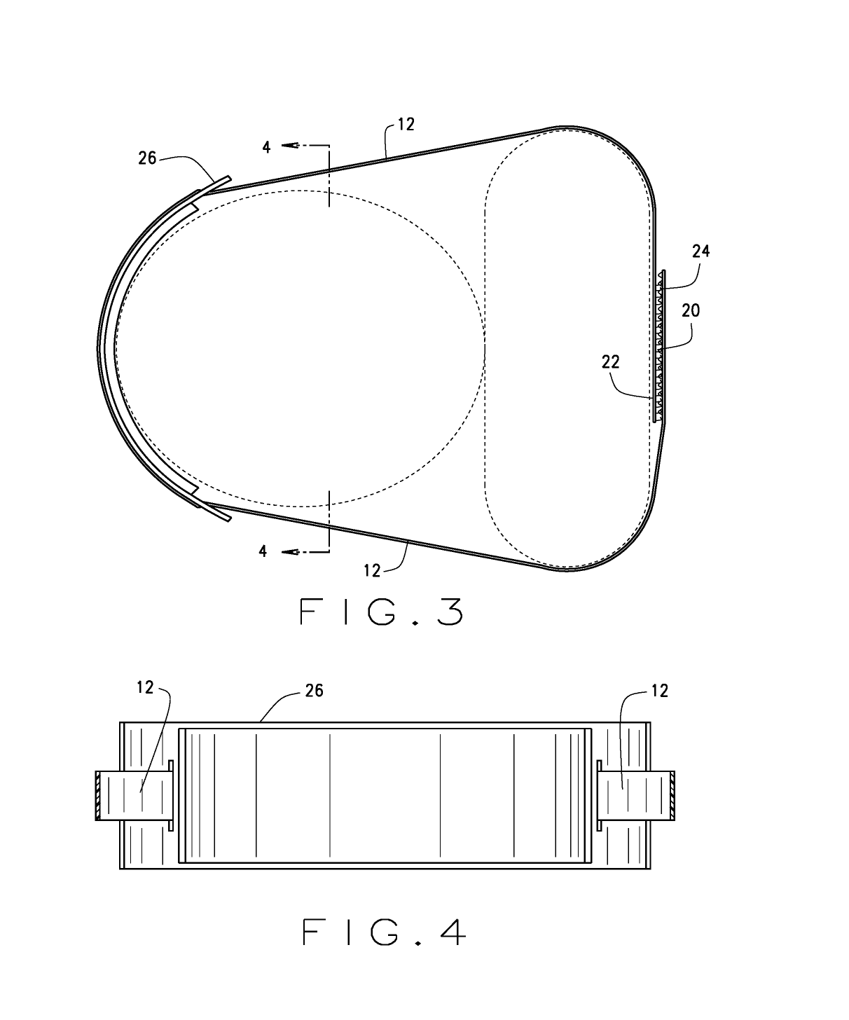

The head support device 10 of the present invention is generally shown in FIG. 1.

It comprises a flexible band or strap member 12. Strap member 12 preferably has a degree of elasticity so that it may stretch slightly. Head support device 10 is employed by a passenger 14 while sitting in a high-backed seat 16 as shown in FIG. 2. Seat 16 may be a plane seat or a train seat which provides a relatively narrow seat portion and is typically adjacent to one or more other seats of similar structure. The gaps between adjacent seats permit strap member 12 to be placed over an upper portion 18 of seat 16. The height at which strap member 12 is placed by the user can be readily adjusted so that it lies even with the forehead of the person. Accordingly, strap member 12 should have a sufficient length to place over and around seat back 18 and provide enough room for the person to place a top of his/her head within the confines of strap member 12 as shown in FIG. 2. The head support device therefore provides support to a person's head while sitting upright in an airplane seat. The elasticity of the strap provides gentle comfort and support to the wearer. The degree of tension placed on the user's head by the appropriate tightening of strap member 12 limits sudden forward jerks of the head that can happen during napping.

Strap member 12 can be made adjustable to provide a tighter or looser fit. Portions of opposing exterior edges of separate ends of strap member 12 may comprise hook-and-loop fastener elements 20 as shown in FIG. 1. Strap member 12 may be open-ended at a portion such that a loose strap portion 22 may overlap a loose strap portion 24 and be re-joined through hook-and-loop fastener elements 20 as necessary to provide an appropriate tightness to the overall strap member 12 as shown in FIG. 3. Another embodiment of the invention may incorporate buckle elements (not shown) to provide the ability to tighten strap member 12.

An embodiment of the invention incorporates an eye mask 26. Eye mask 26 can be integrated directly into strap member 12. Another embodiment of the invention can adapted so that a front portion of the strap member that opposes the wearer's forehead be provided with an attachment element (such as hook-and-loop fasteners) so that a separate sleep mask may be attached.

Another embodiment of the invention provides for a forehead pad positioned similarly to element 26 as shown in FIG. 6. The forehead pad can be lined on its interior facing surface with soft, non-abrasive material so as not to irritate the user's forehead during use. The forehead pad can be provided at its ends with openings to receive strap member 12 in sliding relationship so that the user can adjust the placement of the forehead pad along strap member 12 where the forehead pad moves independently of the strap member. In this fashion, if the user wishes to tilt her head to the side, the forehead pad can be moved along strap member 12 without having to move the strap itself.

The head support device of the invention is very simple to deploy and need only be placed over and around the seat back. No extra attachment elements are required and when not in use, can simply be lifted off the seat back. The strap can be easily and compactly folded up for ready storage in one's traveling bag or purse.

Although the invention has been disclosed with reference to various particular embodiments, it is understood that equivalents may be employed and substitutions made herein without departing from the scope of the invention.

What is claimed is:

1. A head support apparatus for use in a seat, the head support apparatus comprising a flexible strap member having sufficient length to encircle around a seat back while engaging a user's head, the strap member comprising a forehead engaging member, the strap member being adjustable in length to vary a tension placed on the user's head while sitting in the seat.

2. The head support apparatus of claim 1 in which the strap member is elastic.

3. The head support apparatus of claim 1 in which the strap member has separable ends whereby adjustability of the head support apparatus is effected through connection of the separable ends.

4. The head support apparatus of claim 3 in which the separable ends of the strap member are connected by a buckle member.

5. The head support apparatus of claim 3 in which the separable ends of the strap member are connected by hook and loop fasteners.

6. The head support apparatus of claim 1 in which the forehead engaging member comprises an eye mask element.

7. The head support apparatus of claim 1 in which the forehead engaging member is received in sliding relationship with the strap member.

8. The head support apparatus of claim 1 in which a surface of the forehead engaging member comprises a soft, non-abrasive material.

9. The head support apparatus of claim 1 in which the forehead engaging member is removable from the strap member.
